(19) United States
(12) Patent Application Publication
(10) Pub. No.: US 2025/0099715 A1
(43) Pub. Date: Mar. 27, 2025

(54) Delivery Device for Probe With Asymmetric Tip

(71) Applicant: Becton, Dickinson and Company, Franklin Lakes, NJ (US)

(72) Inventor: Yiping Ma, Layton, UT (US)

(21) Appl. No.: 18/893,219

(22) Filed: Sep. 23, 2024

Related U.S. Application Data
(60) Provisional application No. 63/584,797, filed on Sep. 22, 2023.

(51) Int. Cl. A61M 25/01; A61M 25/00

(52) U.S. Cl. CPC A61M 25/0105; A61M 2025/0098

(57) ABSTRACT

Provided herein is a probe delivery device for advancing a probe into a vascular access device. The probe delivery device includes an outer housing, a connector, a shield assembly, and a probe assembly. The shield assembly includes a shield sleeve movable within an inner volume of the housing and a shield advancer coupled to the proximal end of the shield sleeve, the shield advancer having a shield handle configured to slide along an outer surface of the housing to move the shield sleeve distally from a first sleeve position to a second sleeve position. The probe assembly includes a probe movable within the shield sleeve and a probe advancer coupled to the proximal end of the probe, the probe advancer having a probe handle configured to slide along an outer surface of the housing to move the probe distally from a first probe position to a second probe position.

### CROSS-REFERENCE TO RELATED APPLICATION

The present application claims priority to U.S. Provisional Patent Application No. 63/584,797 entitled “Delivery Device for Probe with Asymmetric Tip” filed Sep. 22, 2023, the entire disclosure of which is hereby incorporated by reference.

### BACKGROUND OF THE INVENTION

### Field of the Invention

The present disclosure relates generally to instrument delivery devices for use with intravenous (IV) catheters and, more specifically, to delivery devices for probes with an asymmetric tip.

### Description of Related Art

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The catheter is typically provided as part of a catheter assembly that also includes a catheter hub or adapter, along with other connectors or extensions that provide for connection of external devices to the catheter assembly.

It is recognized that a catheter may be indwelling within the patient for short term (days), moderate term (weeks), or long term (months to years), and that when indwelling IV catheters are maintained within the patient's vasculature, they are likely to become occluded. Once an IV catheter device is occluded, it may no longer be possible to use the IV catheter device to infuse fluids or withdraw blood. In such cases, the IV catheter device may be replaced. Yet, replacing an IV catheter device is burdensome for the patient and increases costs. To address such issues, instrument delivery devices have been developed that can advance and insert a probe into the indwelling IV catheter, with the probe used to reposition the distal end of the IV catheter relative to the occlusion. For example, some instrument delivery devices may employ an asymmetric probe that can be inserted through the catheter and adjacent the catheter's distal opening, with the probe configured with a shaped portion that can lift, advance, retract, or swivel the distal end of the IV catheter to thereby reposition the catheter within the patient's vasculature. This repositioning can move the IV catheter relative to the wall or other anatomy of the vasculature and relative to any obstructions such as a thrombus/occlusion that may have formed. By repositioning the catheter, the probe prolongs the patency of the catheter, including facilitating the collection of a blood sample through a long-dwelling catheter.

While such asymmetric probes are effective in repositioning an IV catheter relative to the wall or other anatomy of the vasculature and relative to any obstructions that may have formed, it is recognized that the shaped portion of these probes can lead to difficulty in introducing them into the IV catheter. For example, asymmetric probes with a V-shaped tip have been shown to be effective to move the catheter tip away from the vein wall to facilitate blood draw success, but the geometry of the V-shaped tip makes the probe susceptible to getting caught when being advanced into the catheter. As one example, the V-shaped tip may get caught on a metal wedge included on a catheter hub or adapter from which the catheter extends. The V-shaped tip may also get caught on another obstruction, whether at a location along the length of the catheter tube or within a housing of the instrument delivery device.

Accordingly, it is desired to provide a probe delivery device useable with an indwelling IV catheter that allows clinicians to advance an asymmetric probe into and through the indwelling IV catheter, while reducing or eliminating the chances of the probe becoming caught on an obstruction in the catheter assembly.

### SUMMARY OF THE INVENTION

Provided herein is a probe delivery device for advancing an asymmetric probe into a vascular access device. The probe delivery device includes an outer housing defining an inner volume and having a proximal end and a distal end and a connector positioned at the distal end of the outer housing and configured to mate with an access connector of the vascular access device. The probe delivery device also includes a shield assembly comprising a shield sleeve arranged longitudinally within the inner volume and movable therein, the shield sleeve having a distal end and a proximal end, and a shield advancer coupled to the proximal end of the shield sleeve, the shield advancer having a shield handle configured to slide along an outer surface of the housing. The probe delivery device further includes a probe assembly comprising a probe positioned within the shield sleeve and movable therein, the probe having a distal end and a proximal end, and a probe advancer coupled to the proximal end of the probe, the probe advancer having a probe handle configured to slide along an outer surface of the housing. The shield advancer is configured to move relative to the outer housing, with a distal movement of the shield advancer moving the shield sleeve from a first sleeve position, in which a distal end of the shield sleeve is disposed within the outer housing, to a second sleeve position, in which the distal end of the shield sleeve is disposed beyond the distal end of the outer housing and the connector. The probe advancer is configured to move relative to the outer housing, with a distal movement of the probe advancer moving the probe from a first probe position, in which a distal end of the probe is disposed within the outer housing, to a second probe position, in which the distal end of the probe is disposed beyond the distal end of the outer housing and the connector.

In some embodiments, the shield handle is positioned on the outer housing proximal from the probe handle, and wherein the distal movement of the shield handle causes a corresponding distal movement of the probe handle.

In some embodiments, the shield handle is movable distally along the outer housing by a first distance, and wherein the probe handle is movable distally along the outer housing by a second distance greater than the first distance.

In some embodiments, when the shield sleeve and the probe are in their respective second sleeve position and second probe position, the distal end of the probe extends out further distally from the distal end of the shield sleeve, such that the distal end of the probe is positioned outside of the shield sleeve.

In some embodiments, the probe handle is configured to rotate relative to the outer housing when the probe is in the second probe position, with rotation of the probe handle causing the probe to rotate within the catheter when the probe is in the second position.

In some embodiments, the probe handle is configured to rotate in a unidirectional manner or a bidirectional manner.

In some embodiments, the probe advancer comprises a coupling element that engages the probe handle with the proximal end of the probe.

In some embodiments, each of the proximal end of the probe and the coupling element comprise a magnet or a magnetic material, to magnetically couple the probe with the coupling element, such that distal movement or rotation of the probe handle causes a corresponding distal movement or rotation of the probe.

In some embodiments, the outer housing contains a plurality of markings thereon, the plurality of markings including at least a first marker indicating when the shield handle has been advanced distally along the outer housing by the first distance.

In some embodiments, the plurality of markings includes at least one additional marker indicating when the probe handle has been advanced distally along the outer housing by at or near the second distance.

In some embodiments, the probe delivery device further includes a fluid flow preventing seal positioned in or adjacent the connector and configured to prevent a transfer of fluid between the vascular access device and the inner volume of the outer housing.

In some embodiments, the shield advancer and the probe advancer comprise an integral advancer assembly, and wherein each of the proximal end of the shield sleeve and the proximal end of the probe are coupled to the advancer assembly, and wherein the advancer assembly is configured to move relative to the outer housing, with a distal movement of the advancer assembly moving the probe and the shield sleeve from their respective first positions to their respective second positions.

In some embodiments, the probe delivery device further includes a locking mechanism positioned on the connector that locks the advancer assembly to the connector, and wherein the outer housing is rotatable relative to the advancer assembly when locked with the connector, with rotation of the outer housing causing the probe to rotate within the catheter when the probe is in the second position will allow the outer housing to rotate.

In some embodiments, the distal end of the probe comprises a shaped portion for causing a distal end of the catheter to be repositioned as the probe is selectively extended into the catheter.

In some embodiments, the shaped portion is formed of a shape memory material that is straight at an ambient room temperature and forms a bend at body temperature.

In some embodiments, the shield sleeve comprises a rigid sleeve or a flexible sleeve.

Also provided herein is a system for performing a blood draw. The system includes a vascular access device comprising a catheter adapter having a proximal end and a distal end and defining a lumen therein and a catheter that extends distally from the catheter adapter, the catheter having a proximal end and a distal end. The system also includes a probe delivery device, wherein, with the probe in the second probe position, the distal end of the probe is disposed adjacent the distal end of the catheter.

In some embodiments, the catheter adapter comprises a wedge positioned within the lumen adjacent the distal end of the catheter adapter, the wedge configured to secure a proximal end of the catheter to the catheter adapter, and wherein with the shield sleeve in the second sleeve position, the distal end of the shield sleeve is disposed distal to the wedge.

In some embodiments, the probe delivery device is configured to advance the probe so that the distal end thereof is adjacent the distal end of the catheter.

In some embodiments, an extension set coupled to the proximal end of the shield sleeve, with the shield sleeve provides a fluid flow path between the vascular access device and the extension set.

### BRIEF DESCRIPTION OF THE DRAWINGS

FIG. 1 is a side view of a catheter assembly and an associated probe delivery device useable therewith, according to an embodiment described herein;

FIG. 2 is a side cross-section view of the catheter assembly of FIG. 1, taken along line 2-2;

FIG. 3 is a side cross-section view of the probe delivery device of FIG. 1, with the probe delivery device in a first configuration;

FIG. 4 is a side cross-section view of the probe delivery device of FIG. 1, with the probe delivery device in a second configuration;

FIG. 5 shows an isolated view of an asymmetric probe included in the probe delivery device of FIG. 1, according to an embodiment described herein;

FIG. 6 is a side view of a catheter assembly and an associated probe delivery device useable therewith, according to another embodiment described herein;

FIG. 7 is a side cross-section view of the probe delivery device of FIG. 6, with the probe delivery device in a first configuration; and

FIG. 8 is a side cross-section view of the probe delivery device of FIG. 6, with the probe delivery device in a second configuration.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

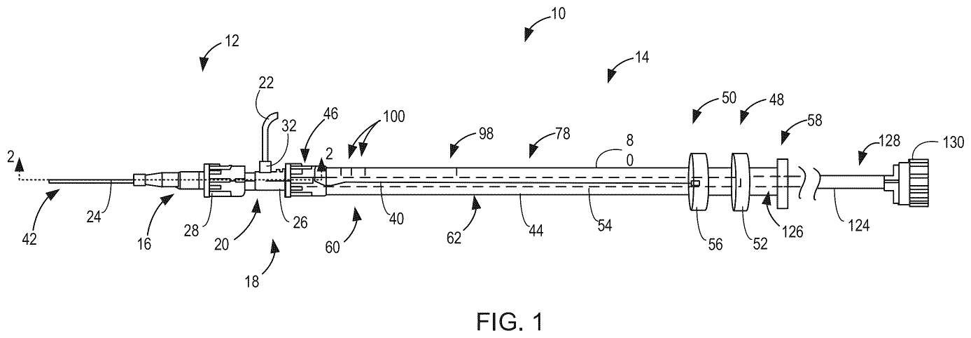

Referring now to FIGS. 1-5, shown is a non-limiting embodiment of a catheter system 10 that includes a vascular access device, in the form of a catheter assembly 12, and an associated instrument delivery device 14, with the instrument delivery device 14 configured to facilitate delivery of an instrument into an in-dwelling catheter of the catheter assembly 12, such as may be desirable for blood collection from the patient. According to a non-limiting embodiment, the catheter assembly 12 includes a catheter hub 16 and an extension set 18 (optionally) that includes a catheter connector 20 and (optionally) extension tubing 22. The catheter hub 16 receives a catheter 24 and is coupled to the catheter connector 20. In one aspect or embodiment, the catheter hub 16 is the AccuCath™ catheter system commercially available from Becton, Dickinson and Company. The catheter 24 may be employed as a peripheral intravenous catheter, a midline catheter, or a peripherally-inserted central catheter, and the catheter 24 may be formed of any suitable material and may be of any useful length, as known to those of skill in the art.

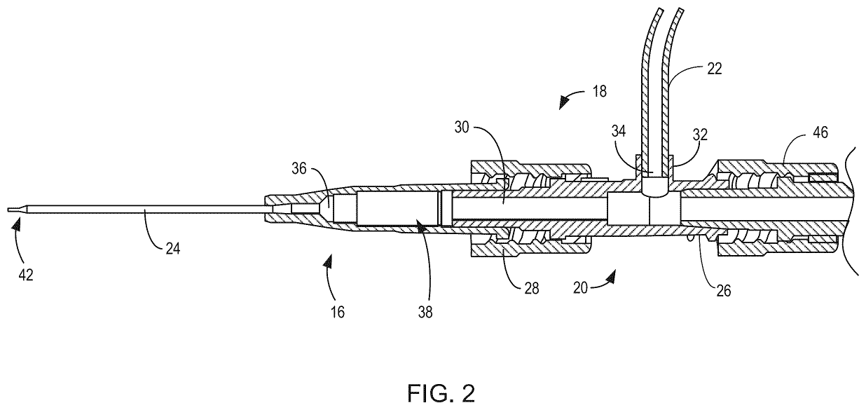

The catheter connector 20 is configured to be placed in contact with the skin surface of a patient at or near an insertion site of catheter 24. While not shown, it is recognized that the catheter connector 20 may include stabilization features (e.g., wings) thereon that assist with retaining the catheter connector 20 in place on the skin of a patient. The catheter connector 20 includes a proximal port with a coupler 26 and a distal port with a distal coupler 28, and defines at least one lumen 30 (FIG. 2) extending through or otherwise in fluid communication with the couplers 26 and 28. The proximal coupler 26 and/or the distal coupler 28 can be, for example, male or female luer locks and/or any other suitable coupler, with the non-limiting embodiment of FIGS. 1-5 illustrating the proximal coupler 26 as a female luer connector and the distal coupler 28 as a male luer connector. The proximal coupler 26 can be physically and fluidically coupled to the instrument delivery device 14 to enable introduction of a probe into the catheter and performing of a subsequent blood draw, as will be explained in further detail below. The distal coupler 28 can be physically and fluidically coupled to catheter hub 16 such that the lumen 30 of the catheter connector 20 is at least selectively in fluid communication with the catheter 24.

The catheter connector 20 may also include and/or define one or more additional ports, such as a side port 32. Side port 32 defines a lumen 34 that is in fluid communication with the lumen 30 between distal coupler 28 and proximal coupler 26. As such, side port 32 can provide access to the lumen 30 between distal coupler 28 and proximal coupler 26, which in turn can provide access to the catheter hub 12 (and catheter 24) that is coupled to the distal coupler 28. In some embodiments, the arrangement of side port 32 can be such that the catheter connector 20 forms, for example, a Y-connector or a T-connector. As previously indicated, it is recognized that extension set 18 may include extension tubing 22 that is connected to side port 32 and in fluid communication with the lumen 34 of the side port 32. In some embodiments, the side port 32 and/or the extension tubing 22 can be and/or can form at least a portion of a fluid line that can be used to deliver fluid, remove fluid, flush fluid, and/or the like.

As shown in FIG. 2, according to some aspects or embodiments, the catheter hub 16 may include a metal wedge 36 positioned within a lumen 38 formed therethrough. The wedge 36 is positioned within the lumen 38 and at a distal end of the catheter hub 16, such that the wedge 36 may be seated within the lumen 38. The wedge 36 is configured to receive a proximal end of the catheter 24 such that the catheter 24 is secured to the wedge 36, thereby anchoring the catheter 24 to the catheter hub 16.

The instrument delivery device 14 of system may be operated to introduce an instrument into the catheter assembly 12 and into the vasculature of the patient. In some embodiments, and as referred to hereafter, the instrument delivery device 14 may comprise a probe delivery device (“probe delivery device 14”) that introduces a probe 40 through the catheter hub 16 and into the indwelling catheter 24. In some embodiments, the probe 40 may be an asymmetric probe having a bent or shaped distal end configured to reposition a distal end or tip 42 of the catheter 24 while the catheter 24 remains inserted in a patient's vasculature, thereby moving the catheter tip 42 relative to the wall or other anatomy of the vasculature and relative to any obstructions such as a thrombus/occlusion that may have formed and provide for a subsequent blood draw to be performed.

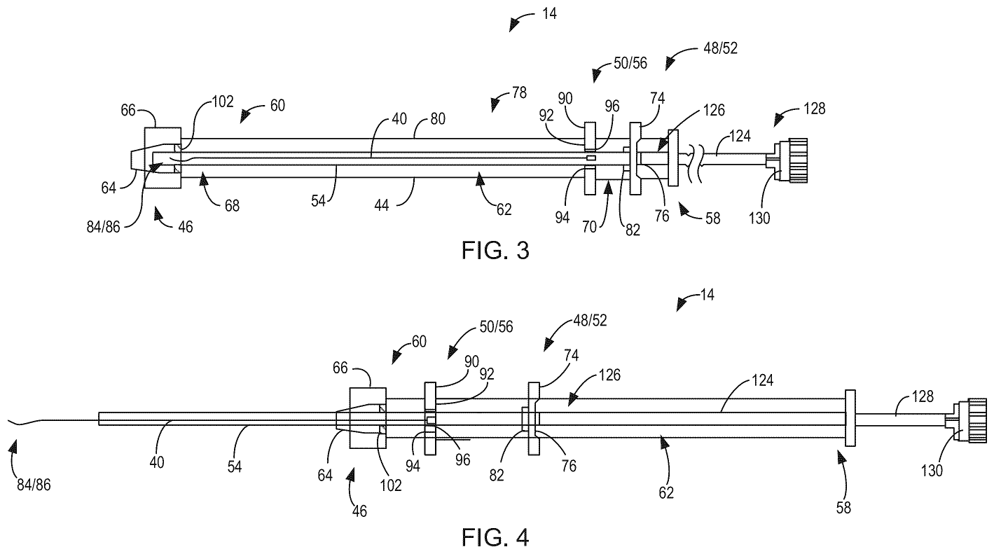

As shown in FIG. 1 and in FIGS. 3-5, according to a non-limiting embodiment, the probe delivery device 14 includes at least a housing 44, a connector 46, a shield assembly 48, and a probe assembly 50—with the shield assembly 48 including a shield advancer 52 and a shield sleeve 54 and the probe assembly 50 including a probe advancer 56 and probe 40. As will be described in further detail below, the probe 40 is moveable within the housing 44 so as to provide for advancement of a portion of the probe 40 from a first or retracted position inside the housing 44 (FIG. 3) to a second or advanced position outside of the housing 44 (FIG. 4), such that a distal end thereof may be routed into the catheter assembly 12, while the shield sleeve 54 is also movable from a first position inside the housing 44 (FIG. 3) to a second position outside of the housing 44 (FIG. 4), with the shield sleeve 54 preventing a shaped portion at the distal end of the probe 40 from becoming caught or lodged within the catheter assembly (or within housing 44) as it is advanced from its first position to its second position.

The housing 44 of blood draw device 14 can be an elongate member having a proximal end 58 and a distal end 60 and defining an inner volume 62. In some embodiments, the housing 44 may be formed of a pair of housing portions that are arranged side-by-side and coupled together to define the inner volume 62. The housing 44 may include one or more features or surface finishes on an outer surface thereof that can be arranged to increase the ergonomics of the blood draw device 14, which in some instances can allow a user to manipulate the blood draw device 14 with one hand (i.e., single-handed use).

The connector 46 of blood draw device 14 is provided at the distal end 60 of the housing 44, with the connector 46 providing for reversible coupling of the blood draw device 14 to catheter assembly 12, such as via proximal coupler 26 as shown in FIG. 1. The connector 46 is exemplified as a male luer connector that mates with the female luer connector of proximal coupler 26 on connector 20, with the male luer connector of connector 46 having an elongated member 64 surrounded by a rotating collar 66. The rotating collar 66 may be rotated to threadingly engage the male luer connector to the female luer connector of proximal coupler 26. However, it is appreciated that alternative embodiments of probe delivery device 14 may include a connector 46 of another type to secure probe delivery device 14 to catheter assembly 12, including luer slip connections, clips, blunt plastic cannulae, blunt metal cannulae, hybrid luers (e.g., with a cannula), friction fits, and the like.

As indicated above, the shield assembly 48 of blood draw device 14 includes a shield advancer 52 and a shield sleeve 54. The shield sleeve 54 is positioned within the inner volume 62 of housing 44 and extends generally along a length of the housing 44. The shield sleeve 54 is configured as an elongated, tubular member having a distal end 68 and a proximal end 70 and defining a shield lumen 72 therein. The shield sleeve 54 may be formed of any rigid or flexible material, so long as the material is sufficiently rigid so as to resist buckling. In non-limiting embodiments, shield sleeve 54 is formed of a material that provides resistance to buckling, such as polyethylene, polypropylene, nylon, polyurethane, polyimide, and the like. In some embodiments, the distal end 68 of shield sleeve 54 may include fenestrations (not shown) formed therein that allow for a higher flow rate of blood draw (or infusion) through catheter assembly 12 (and delivery device 14).

The shield advancer 52 includes a first portion 74 and a second portion 76. The first portion 74 is movably disposed along an outer surface 78 of the housing 44 and the second portion 76 is movably disposed within the inner volume 62 of the housing 44. In some embodiments, the arrangement of the shield advancer 52 and the housing 44 is such that a connecting portion (not shown) of the shield advancer 52 that joins the first and second portions 74, 76 is seated within a slot 80 formed in the upper surface 78 of the housing 44—the slot 80 generally extending between the proximal and distal ends 58, 60 of the housing 44. As the first and second portions 74, 76 are joined together, movement of the first portion 74 along the outer surface 78 of the housing 44 results in a corresponding movement of the second portion 76 within the inner volume 62.

As shown in FIGS. 3 and 4, the first portion 74 of the shield advancer 52 may be configured as a (shield) handle (hereafter “handle 74”) that is positioned on the housing 44. In some embodiments, the handle 74 may be configured as an annular member that generally encircles the housing 44 and is in contact with the outer surface 78 thereof. The handle 74 is engageable by a user such that it may be pushed distally along the outer surface 78 of the housing 44 and relative thereto.

As shown in FIGS. 3 and 4, the second portion 76 is movably disposed within the inner volume 62 of the housing 44 and includes an attachment member 82 (e.g., a seat or opening) by which the shield sleeve 54 may be secured to the shield advancer 52, with the attachment member 82 configured to grip or retain the proximal end 70 of the shield sleeve 54. Due to the shield sleeve 54 being retained by attachment member 82 of second portion 76, movement of the shield advancer 52 relative to housing 44 causes a corresponding movement of the shield sleeve 54 relative to the housing 44. In this manner, the distal end 68 of the shield sleeve 54 can be selectively moved out of or back into the inner volume 62 of the housing 44 as desired, such as advancing the distal end 70 of the shield sleeve 54 out of the housing 44 when the probe delivery device 14 has been coupled to the catheter assembly 12 and it is desired to advance the probe 40 into the catheter 24.

As indicated above, the probe assembly 50 of probe delivery device 14 includes a probe advancer 56 and a probe 40. The probe 40 may be constructed as a wire formed of a metallic or other suitable material, with the wire having a diameter that provides for positioning of the wire within the catheter 24. The probe 40 is positioned within the lumen 72 of shield sleeve 54 and, as described in further detail below, may be moved relative to the shield sleeve 54 via operation of the probe advancer 56, such that a distal end 84 of probe 40 may be advanced out from the distal end 68 of shield sleeve 54.

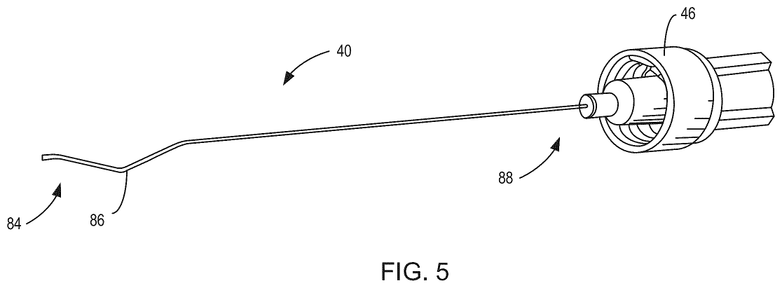

As indicated above, according to aspects of the disclosure, the probe 40 may be configured as an asymmetric probe having a bent or shaped distal end 84 configured to reposition a distal end 42 of the catheter 24 when the probe 40 is advanced into and/or rotated within catheter 24. FIG. 5 provides an example of how probe 40 may be configured to cause this repositioning. In FIG. 5, probe 40 is shown as having a distal end 84, a shaped portion 86 and a proximal end 88. Shaped portion 86 should be construed as a length of probe 40 positioned at or towards distal end 84 that has a shape that deviates from a longitudinal axis of proximal end 88 and that generally retains this shape when distal end 84 is positioned proximate to or at a distal end 42 of catheter 24. In the illustrated embodiment, shaped portion 86 is in the form of a v-shaped length of probe 40 positioned between proximal end 88 and distal end 84, with the v-shaped length adjacent the distal end 84 of probe 40. In other embodiments, it is recognized that shaped portion 86 could instead be configured to have an inverted v shape or to have a curved w shape, according to other non-limiting examples.

In some embodiments, shaped portion 86 may flex, flatten, or otherwise adapt its shape as it is advanced through catheter 24 and, when positioned at or near the distal end 42 of catheter 24, may generally retain its shape to thereby act on the distal end 42 of catheter 24 causing it to be repositioned. As stated above, the shape of probe 40 would necessarily need to adapt to the confines of catheter 24 as probe 40 is advanced. Yet, even with such adaptations, shaped portion 86 is configured to substantially retain its shape relative to distal end 84 and proximal end 88. In other embodiments, it is recognized that the shaped portion 86 may be formed of a shape memory material that is straight at an ambient room temperature and forms a bend at body temperature.

Similar to shield advancer 52, probe advancer 56 is also formed to include a first portion 90 and a second portion 92. The first portion 90 is movably disposed along an outer surface 78 of the housing 44 and the second portion 92 is movably disposed within the inner volume 62 of the housing 44. In some embodiments, the arrangement of the probe advancer 56 and the housing 44 is such that a connecting portion (not shown) of the probe advancer 56 that joins the first and second portions 90, 92 is seated within slot 80 formed in the outer surface 78 of the housing 44—the slot 80 generally extending between the proximal and distal ends 58, 60 of the housing 44. As the first and second portions 90, 92 are joined together, movement of the first portion 90 along the outer surface 78 of the housing 44 results in a corresponding movement of the second portion 92 within the inner volume 62.

As shown in FIGS. 3 and 4, the first portion 90 of the probe advancer 56 may be configured as a (probe) handle (hereafter “handle 90”) that is positioned on the housing 44. In some embodiments, the handle 90 may be configured as an annular member that generally encircles the housing 44 and is in contact with the outer surface 78 thereof. The handle 90 is engageable by a user such that it may be pushed distally along the outer surface 78 of the housing 44 and relative thereto, with the probe handle 90 positioned distally from the shield handle 74.

As shown in FIGS. 3 and 4, the second portion 92 of probe advancer 56 is movably disposed within the inner volume 62 of the housing 44 and is configured such that at least a portion thereof is positioned adjacent or about the shield sleeve 54. According to aspects of the disclosure, the second portion 92 includes a coupling element 94 thereon that is configured to interact with probe 40 such that movement of the probe advancer 56 relative to housing 44 causes a corresponding movement of the probe 40 relative to the housing 44. In an exemplary embodiment, the coupling element 94 may be provided as a ring or other shaped member that is positioned about the shield sleeve 54 and is configured as a magnetic element (i.e., formed of a magnetic material or including a magnet thereon) that interacts with a magnetic element 96 on the proximal end of probe 40, such that the coupling element 94 is magnetically coupled with the probe 40. In this manner, the distal end 84 of the probe 40 can be selectively moved out of or back into the inner volume 62 of the housing 44 as desired, such as advancing the distal end 84 of the probe 40 out of the housing 44 when the probe delivery device 14 has been coupled to the catheter assembly 12 and it is desired to advance the probe 40 into the catheter 24.

With the construction of probe delivery device 14 as described above (i.e., with shield advancer 52 proximal from probe advancer 56 on housing 44), advancement of the shield advancer 52 distally along housing 44 will cause a corresponding advancement of the probe advancer 56 distally along housing 44, such that both the shield sleeve 54 and the probe 40 are advanced together relative to housing 44. Additionally, and when desired, the probe advancer 56 may be further advanced distally along the housing 44 after shield advancer 52 has reached a desired location along housing 44, such that the probe 40 may be advanced further distally while the shield sleeve 54 remains in place—i.e., the probe handle 90 is movable distally along the housing 44 by a (second) distance greater than a (first) distance that the shield handle 74 is movable distally along the housing 44. Accordingly, the distal end of the probe 40 may be extended out further distally from the distal end 68 of the shield sleeve 54, such that the distal end 84 of the probe 40 is positioned outside of the shield sleeve 54.

According to aspects of the disclosure, in addition to the probe advancer 56 being movable along the housing 44, the probe advancer 56 is further configured to rotate relative to the housing 44, so as to provide for rotation of the probe 40 and repositioning of the distal end 42 of the catheter 24 away from vein wall, thrombus, or other potential feature on the vein that is preventing blood draw success. In particular, with the probe advancer 56 advanced distally and the probe 40 in its second position, the probe handle 90 may be rotated relative to the housing 44, with the magnetic coupling between the probe advancer 56 (i.e., coupling element 94 thereof) and the proximal end 88 of probe 40 transferring a rotation of the probe handle 90 to the probe 40, to cause rotation of the probe 40 and the asymmetric tip thereof in a desired angular direction. According to embodiments, rotation of the handle 90 may be enabled in just one direction or rotation may be enabled in both directions (for back and forth rotation of the probe tip).

According to some aspects of the disclosure, and as shown in FIG. 1, the housing 44 may include one or more position markers thereon that indicate shield sleeve 54 and/or probe 40 positioning relative to the components of the catheter assembly 12. According to a non-limiting embodiment, a first position marker 98 may be provided on the housing 44 as indicia, such as a colored region, text, or a symbol. The first position marker 98 may be provided on a top surface (or about an entire outer surface) of the housing 44 and may indicate to an operator when the shield sleeve 54 has advanced distally to its second location, such that the shield sleeve 54 extends into or distally past the wedge 36 (in catheter hub 16). With the shield advancer 52 aligned with the first position marker 98 on housing 44, the shield sleeve 54 will be in the second position, with the distal end 68 thereof extending into or distally past the wedge 36, and thereby ensuring that the probe 40 will not become caught on the wedge 36. One or more second position markers 100 may also be provided on the housing 44 as indicia, such as a colored region, text, or a symbol. The second position markers 100 may be provided on a top surface (or about an entire outer surface) of the housing 44 and may indicate to an operator when the probe 40 has advanced distally near or to its second location, such that the probe 40 is positioned near or at the distal tip 42 of catheter 24. In some embodiments, second position markers 100 may indicate when the distal end 84 of the probe 40 is within 2 inches, 1.25 inches, 1.0 inches, or at the distal tip 42 of the catheter 23. With the probe advancer 56 aligned with one of the second position markers 100 on housing 44, the probe 40 will be at or near its second position, such that the distal end 84 thereof is positioned at/adjacent the distal tip 42 of catheter 24, so as to enable positioning of the distal tip 42 of catheter 24 away from a vein wall or thrombus/occlusion thereat.

According to additional aspects of the disclosure, and as shown in FIGS. 3 and 4, probe delivery device 14 may include a fluid flow preventing septa or seal member 102 that prevents unwanted fluid transfer between the catheter assembly 10 and the probe delivery device 14. That is, a flow preventing seal 102 may be provided in or adjacent the connector 46 of probe delivery device 14 that is configured to prevent an unwanted transfer of fluid between the catheter assembly 10 and the inner volume 62 of the housing 44. In some embodiments, the seal member 102 is formed within the connector 46 (i.e., within a lumen thereof). The seal member 102 may be formed of a flexible, elastomeric material according to one embodiment, and includes an opening 104 formed therein through which the shield sleeve 54 passes. Based on the sizing of the opening 104, a seal is formed between the seal member 102 and the shield sleeve 54 as the shield sleeve and probe are advanced through the opening 104 when the shield sleeve 54 and probe 40 is moved to the second position. In some embodiments, a lubricant (e.g., silicone-based lubricant) can be added to the opening 104 of seal member 102 or to the outer surface of shield sleeve 54 to reduce friction between the shield sleeve 54 and the seal member 102.

In usage of the probe delivery device 14, and upon connection thereof to a catheter assembly 12, an operator may push the shield advancer 52 distally along the housing 44. Pushing of the shield advancer 52 causes the shield sleeve 54 to advance within the housing 44 and, in some embodiments, the shield advancer 52 may be distally advanced until the shield sleeve 54 has been moved forward past the wedge 36 of catheter hub 16 or another known obstruction. Pushing of the shield advancer 52 distally along the housing 44 also results in the probe advancer 56 being pushed distally along the housing 44 due to the proximal positioning of probe advancer 56 relative to shield advancer 52, with pushing of the probe advancer 56 causing the probe 40 to advance distally within the housing 44 and within shield sleeve 54. Upon the shield advancer 52 being moved distally to its desired location, the operator may continue pushing the probe advancer 56 distally along the housing 44, so as to further advance the probe 40 through the shield sleeve 54 and further into catheter 24. The probe advancer 56 may be distally advanced until the probe 40 reaches the area of catheter tip 42. Once there, the probe advancer 56 can be rotated about the housing 44 to move the asymmetric tip (i.e., shaped portion 86) of the probe 40, with the probe advancer 56 being rotated uni-directionally to rotate the tip in a consistent angular direction or being rotated bi-directionally to rotate the tip back and forth. Both motions may move the catheter tip 42 away from a vein wall, thrombus, or other potential feature on the vein that is preventing blood draw success.

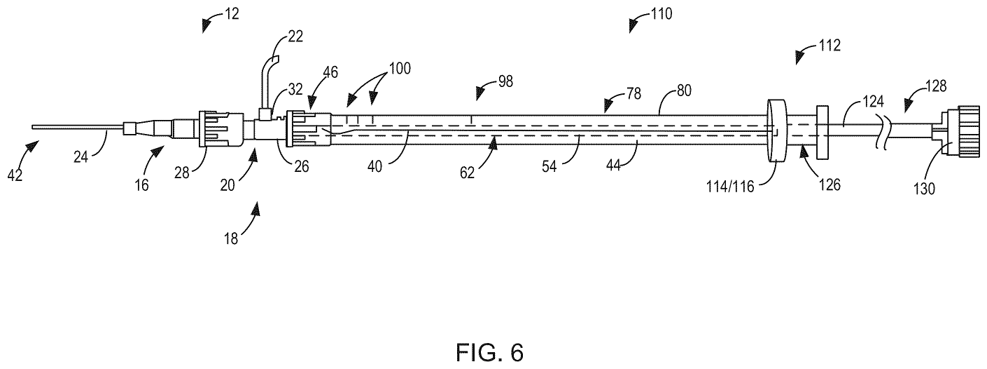

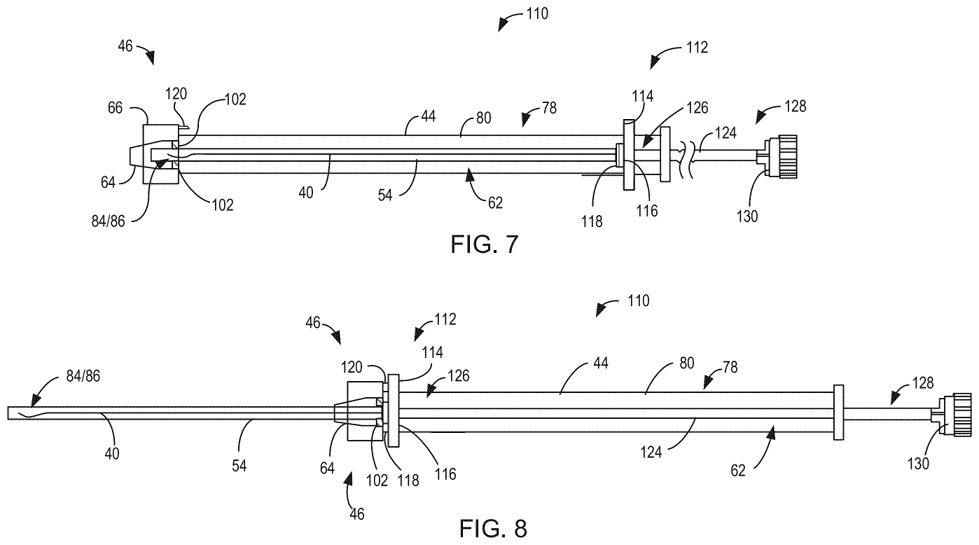

Referring now to FIGS. 6-8, a probe delivery device 110 is shown in accordance with another embodiment of the disclosure. The probe delivery device 110 of FIGS. 6-8 differs from the probe delivery device 14 of FIGS. FIGS. 1-5 in that the separate shield advancer 52 and probe advancer 56 of device are replaced by a single, integral advancer assembly 112 that performs the functions of both the shield advancer 52 and probe advancer 56.

Similar to as previously described, integral advancer assembly 112 may include a first portion 114 and a second portion 116, with the first portion 114 movably disposed along an outer surface 78 of the housing 44 and the second portion 116 movably disposed within the inner volume 62 of the housing 44. The first portion 114 of the advancer assembly 112 may be configured as a handle (hereafter “handle 114”) that is positioned on the housing 44. In some embodiments, the handle 114 may be configured as an annular member that generally encircles the housing 44 and is in contact with the outer surface thereof. The handle 114 is engageable by a user such that it may be pushed distally along the outer surface 78 of the housing 44 and relative thereto. The second portion 116 of advancer assembly 112 is movably disposed within the inner volume 62 of the housing 44 and includes an attachment member 118 (e.g., a seat) by which both the shield sleeve 54 and probe 40 may be secured to the advancer assembly 112, with the attachment member 118 configured to grip or retain the proximal ends of the shield sleeve 54 and probe 40. Due to the shield sleeve 54 and probe 40 being retained by attachment member 118 of second portion 116, movement of the advancer assembly 112 relative to housing 44 causes a corresponding movement of the shield sleeve 54 and probe 40 relative to the housing 44. In this manner, the distal ends 68, 84 of the shield sleeve 54 and probe 40 can be selectively moved out of or back into the inner volume 62 of the housing 44 as desired, such as advancing the distal ends 68, 84 of the shield sleeve 54 and probe 40 out of the housing 44 when the probe delivery device 110 has been coupled to the catheter assembly 12 and it is desired to advance the probe 40 into the catheter 24.

According to aspects of the disclosure, in addition to the advancer assembly 112 being movable along the housing 44, the advancer assembly 112 is further configured to provide for rotation between the housing 44 and the advancer assembly 112, so as to enable rotation of the probe 40 and repositioning of the distal end 42 of the catheter 24 away from vein wall, thrombus, or other potential feature on the vein that is preventing blood draw success. As shown in FIG., connector 46 of probe delivery device 110 includes a locking mechanism 120 positioned on a proximal-facing surface thereof that may engage with the advancer assembly 112 after it has been advanced distally along the housing 44 and brought into engagement with the connector 46. When the advancer assembly 112 is separated from the connector 46, the locking mechanism 120 may be in a locked state that prevents rotation of housing 44 (relative to connector 46). When the advancer assembly 112 is brought into contact/engagement with the connector 46, the handle 114 of advancer assembly 112 actuates locking mechanism 120 to its unlocked state, with the advancer assembly 112 and housing 44 thus being rotatable. In this manner, with the advancer assembly 112 advanced distally to its second position, the probe 40 may be rotated so as to reposition the asymmetric tip (shaped portion 86) thereof in a desired angular direction and thereby also reposition the distal tip 42 of catheter 24.

In usage of the probe delivery device of FIGS. 6-8, and upon connection thereof to a catheter assembly, an operator may push the integral advancer assembly 112 distally along the housing 44. Pushing of the advancer assembly 112 causes the shield sleeve 54 and probe 40 to advance within the housing 44 and, in some embodiments, the advancer assembly 112 may be distally advanced until the shield sleeve 54 and probe 40 have been moved forward past the wedge 36 of catheter hub 16 or another known obstruction and until the distal end 84 of probe 40 (and shield sleeve 54) are advanced to be adjacent the distal tip 42 of indwelling catheter 24. Upon the advancer assembly 112 being moved distally to its desired location, the advancer assembly 112/housing 44 can be rotated to move the asymmetric tip 84 of the probe 40, with the probe advancer 56 being rotated uni-directionally to rotate the tip 84 in a consistent angular direction or being rotated bi-directionally to rotate the tip 84 back and forth. Both motions may move the catheter tip 42 away from a vein wall, thrombus, or other potential feature on the vein that is preventing blood draw success.

According to additional aspects of the disclosure, in either of the delivery devices 14, 110 of FIGS. 1-5 and 6-8, a blood draw may be performed with the probe delivery device 14, 110 coupled with catheter assembly 12 and the probe 40 at its second position—i.e., with the shaped portion 86 of the probe 40 adjacent the distal tip 42 of catheter 42, so as to reposition the catheter tip 42. In providing for the blood draw, an extension set or blood collection device may be connected at the proximal end of delivery device 14, 110. In some embodiments, the extension set includes a secondary catheter 124 that is placed in fluid communication with the shield sleeve 54, with the shield sleeve 54 providing a fluid flow path between the catheter assembly 12 and the secondary catheter 124. A distal end 126 of the secondary catheter 124 may be secured to the second section 76 of shield advancer 52 (or second section 116 of advancer assembly 112) so as to be aligned with shield sleeve 54. In some embodiments, secondary catheter 124 may be secured to the second section 76 via attachment member 82, 118, which may be an opening, in alignment with the shield sleeve 54. A proximal end 128 of the secondary catheter 124 may include thereon a connector 130 (e.g., luer connector) to which a blood collection device (not shown) such as an evacuated syringe may be connected, to enable performing of a blood draw.

Beneficially, aspects of the disclosure thus provide a probe delivery device useable with a catheter assembly having a catheter hub and an indwelling IV catheter, with the probe delivery device allowing clinicians to advance an asymmetric probe into and through the indwelling IV catheter while reducing or eliminating the chances of the probe becoming caught on an obstruction in the catheter assembly. The probe delivery device includes a shield sleeve within which the probe is positioned, with each of the shield sleeve and the probe configured to be advanced into the catheter assembly. The shield sleeve may prevent the probe from being caught on a wedge of the catheter hub and/or another obstruction in the catheter assembly or delivery device.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A probe delivery device for advancing an asymmetric probe into a vascular access device, the probe delivery device comprising:
an outer housing defining an inner volume and having a proximal end and a distal end;
a connector positioned at the distal end of the outer housing and configured to mate with an access connector of the vascular access device;
a shield assembly comprising:
a shield sleeve arranged longitudinally within the inner volume and movable therein, the shield sleeve having a distal end and a proximal end; and
a shield advancer coupled to the proximal end of the shield sleeve, the shield advancer having a shield handle configured to slide along an outer surface of the housing; and
a probe assembly comprising:
a probe positioned within the shield sleeve and movable therein, the probe having a distal end and a proximal end; and
a probe advancer coupled to the proximal end of the probe, the probe advancer having a probe handle configured to slide along an outer surface of the housing;
wherein the shield advancer is configured to move relative to the outer housing, with a distal movement of the shield advancer moving the shield sleeve from a first sleeve position, in which a distal end of the shield sleeve is disposed within the outer housing, to a second sleeve position, in which the distal end of the shield sleeve is disposed beyond the distal end of the outer housing and the connector; and
wherein the probe advancer is configured to move relative to the outer housing, with a distal movement of the probe advancer moving the probe from a first probe position, in which a distal end of the probe is disposed within the outer housing, to a second probe position, in which the distal end of the probe is disposed beyond the distal end of the outer housing and the connector.

2. The probe delivery device of claim 1, wherein the shield handle is positioned on the outer housing proximal from the probe handle, and wherein the distal movement of the shield handle causes a corresponding distal movement of the probe handle.

3. The probe delivery device of claim 1, wherein the shield handle is movable distally along the outer housing by a first distance, and wherein the probe handle is movable distally along the outer housing by a second distance greater than the first distance.

4. The probe delivery device of claim 1, wherein when the shield sleeve and the probe are in their respective second sleeve position and second probe position, the distal end of the probe extends out further distally from the distal end of the shield sleeve, such that the distal end of the probe is positioned outside of the shield sleeve.

5. The probe delivery device of claim 1, wherein the probe handle is configured to rotate relative to the outer housing when the probe is in the second probe position, with rotation of the probe handle causing the probe to rotate within the catheter when the probe is in the second position.

6. The probe delivery device of claim 5, wherein the probe handle is configured to rotate in a unidirectional manner or a bidirectional manner.

7. The probe delivery device of claim 1, wherein the probe advancer comprises a coupling element that engages the probe handle with the proximal end of the probe.

8. The probe delivery device of claim 7, wherein each of the proximal end of the probe and the coupling element comprise a magnet or a magnetic material, to magnetically couple the probe with the coupling element, such that distal movement or rotation of the probe handle causes a corresponding distal movement or rotation of the probe.

9. The probe delivery device of claim 1, wherein the outer housing contains a plurality of markings thereon, the plurality of markings including at least a first marker indicating when the shield handle has been advanced distally along the outer housing by the first distance.

10. The probe delivery device of claim 9, wherein the plurality of markings includes at least one additional marker indicating when the probe handle has been advanced distally along the outer housing by at or near the second distance.

11. The probe delivery device of claim 1, further comprising a fluid flow preventing seal positioned in or adjacent the connector and configured to prevent a transfer of fluid between the vascular access device and the inner volume of the outer housing.

12. The probe delivery device of claim 1, wherein the shield advancer and the probe advancer comprise an integral advancer assembly, and wherein each of the proximal end of the shield sleeve and the proximal end of the probe are coupled to the advancer assembly, and wherein the advancer assembly is configured to move relative to the outer housing, with a distal movement of the advancer assembly moving the probe and the shield sleeve from their respective first positions to their respective second positions.

13. The probe delivery device of claim 12, further comprising a locking mechanism positioned on the connector that locks the advancer assembly to the connector, and wherein the outer housing is rotatable relative to the advancer assembly when locked with the connector, with rotation of the outer housing causing the probe to rotate within the catheter when the probe is in the second position will allow the outer housing to rotate.

14. The probe delivery device of claim 1, wherein the distal end of the probe comprises a shaped portion for causing a distal end of the catheter to be repositioned as the probe is selectively extended into the catheter.

15. The probe delivery device of claim 14, wherein the shaped portion is formed of a shape memory material that is straight at an ambient room temperature and forms a bend at body temperature.

16. The probe delivery device of claim 1, wherein the shield sleeve comprises a rigid sleeve or a flexible sleeve.

17. A system for performing a blood draw, the system comprising:
a vascular access device comprising:
a catheter hub having a proximal end and a distal end and defining a lumen therein; and
a catheter that extends distally from the catheter hub, the catheter having a proximal end and a distal end; and
the probe delivery device of claim 1;
wherein, with the probe in the second probe position, the distal end of the probe is disposed adjacent the distal end of the catheter.

18. The system of claim 17, wherein the catheter adapter comprises a wedge positioned within the lumen adjacent the distal end of the catheter adapter, the wedge configured to secured a proximal end of the catheter to the catheter adapter, and wherein with the shield sleeve in the second sleeve position, the distal end of the shield sleeve is disposed distal to the wedge.

19. The system of claim 18, wherein the probe delivery device is configured to advance the probe so that the distal end thereof is adjacent the distal end of the catheter.

20. The system of claim 17, further comprising an extension set including a secondary catheter and coupled to the proximal end of the shield sleeve, with the shield sleeve provides a fluid flow path between the vascular access device and the secondary catheter.
